(19) United States
(12) Patent Application Publication
(10) Pub. No.: US 2025/0311313 A1
(43) Pub. Date: Oct. 2, 2025

(54) SPIN LIGHT EMITTING DEVICE BASED ON TWO-DIMENSIONAL MATERIALS

(71) Applicant: Yuan LU, Nancy (FR)

(72) Inventor: Yuan LU, Nancy (FR)

(21) Appl. No.: 19/092,217

(22) Filed: Mar. 27, 2025

Related U.S. Application Data
(60) Provisional application No. 63/570,297, filed on Mar. 27, 2024.

(51) Int. Cl. H10D 48/00; H10D 48/40; H10D 62/80; H10N 50/20; H10N 50/85

(52) U.S. Cl. CPC H10D 48/385; H10D 48/40; H10D 62/883; H10N 50/20; H10N 50/85

(57) ABSTRACT

Disclosed is a spin light emitting device based on two-dimensional material. The light emitting device comprises: a two-dimensional structure configured to emit circularly polarized light in response to spin-polarized carrier injection, wherein the two-dimensional structure is a two-dimensional Van der Waals heterostructure; a spin injector configured to inject spin-polarized carriers into the two-dimensional Van der Waals heterostructure, wherein the light emitted by the two-dimensional structure has a circular polarization state determined by the magnetization state of the spin injector; and a magnetization controller configured to change the magnetization state of the spin injector. The spin-based light emitting device emits circularly polarized light or single photons on the basis of two-dimensional material at room temperature without introducing a magnetic field, and has the capability of electrical control.

### CROSS-REFERENCE TO RELATED APPLICATIONS

This application claims the benefit of U.S. Provisional Application No. 63/570,297, filed on Mar. 27, 2024, the disclosure of which is incorporated herein by reference in its entirety and for all purposes.

### TECHNICAL FIELD

The disclosure herein relates to a spin light emitting device based on two-dimensional material.

### BACKGROUND

Spin light emitting diode (spin-LED) has been considered as circularly polarized light source for biomedical applications, three-dimensional (3D) screen and optic communications. Two-dimensional (2D) transition metal dichalcogenides (TMDCs) (MoSe2, WSe2, MoS₂, WS₂) has gained intensive interests to realize spin LED owing to their very long spin relaxation time for electrons and holes. Several groups have demonstrated the successful fabricated 2D spin LEDs, including with in-plane magnetized injector and out-of-plane magnetized injector.

However, for the transition into a practical application, it must meet three crucial criteria: operation at room temperature, no need of magnetic field, and the ability for electrical control. None of current work in the field of spin LED based on two-dimensional material can fulfil the three conditions at the same time.

### SUMMARY

According to an aspect of the disclosure, a light emitting device is provided. The light emitting device comprises: a two-dimensional structure configured to emit light in response to carrier injection, wherein the two-dimensional structure is a two-dimensional Van der Waals heterostructure; a spin injector configured to inject spin-polarized carriers into the two-dimensional Van der Waals heterostructure, wherein the light emitted by the two-dimensional structure has a circular polarization state determined by the magnetization state of the spin injector; and a magnetization controller configured to change the magnetization state of the spin injector.

### BRIEF DESCRIPTION OF FIGURES

By more detailed description of the exemplary embodiments of the present disclosure in combination with the accompanying drawings, the above and other purposes, features and advantages of the present disclosure will become more apparent. In the exemplary embodiments of the present disclosure, the same reference numeral generally represents the same component.

FIG. 1 is a schematic view of the light emitting device of the disclosure.

FIG. 2 is a cross-sectional view of the light emitting device (2D spin-LED) according to an embodiment of the disclosure.

FIG. 3 is a top view of the light emitting device according to the embodiment of the disclosure.

FIG. 4 is a cross-sectional view of the light emitting device (2D spin-LED) according to a variant embodiment of the disclosure.

FIG. 5 is a cross-sectional view of the light emitting device (2D spin-LED) according to another variant embodiment of the disclosure.

FIG. 6 is a cross-sectional view of the light emitting device (2D spin-SPS) according to another embodiment of the disclosure.

FIG. 7 schematically shows the emission principle of valley excitons in two-dimensional TMDC materials.

FIG. 8 illustrates the magnetization switching in the injector Hall-bar structure by spin Hall effect (SHE).

### DETAILED DESCRIPTION

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

In the disclosure, a novel spin light emitting device based on two-dimensional material is provide, which can fulfil the above mentioned three conditions: room temperature operation, no need of magnetic field and electrical control of polarization. The spin light emitting device may be configured as, for example, a 2D spin LED or a 2D single photon source (SPS).

The spintronics technology is used to achieve the light emission with desired circular polarization. By depositing a ferromagnetic layer as a spin injection layer on the top of the LED structure based on two-dimensional materials, spin-polarized carriers (electrons or holes) can be injected into the two-dimensional materials. The spin-polarized electrons (or holes) will undergo quantum transition to recombine with holes (or electrons) according to the optical selection rule, and thus circularly polarized photons will be emitted. The two-dimensional material is capable of emitting photons with circular polarization direction determined by the spin direction of the injected spin-polarized carriers.

In embodiments, a perpendicularly magnetized spin injector of for example Ta/CoFeB is formed on a 2D structure-based LED, for example, a three-layer structure including a layer of hexagonal boron nitride (BN), a layer of WS₂ and another layer of hexagonal boron nitride (BN). The magnetization state of the spin injector is configured to be capable of being electrically switched, in order to control the circular polarization of the emitted light.

The new functionality of the 2D spin LED will allow to develop ultra-compact spin/photon helicity converters for applications including cancer detection, 2D single photon source for quantum cryptography and quantum computing.

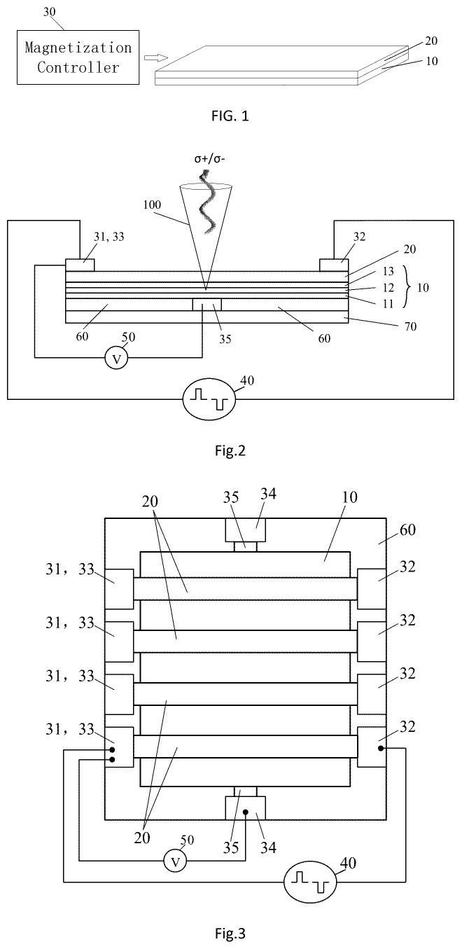

FIG. 1 is a schematic view of the spin light emitting device of the disclosure.

As shown in FIG. 1, the light emitting device includes a two-dimensional (2D) structure 10, a spin injector 20 and a magnetization controller 30.

The magnetization controller 30 is configured to change the magnetization state of the spin injector 20.

The spin injector 20 is configured to inject spin-polarized carriers into the two-dimensional (2D) structure 10, which is a two-dimensional (2D) Van der Waals (VdW) heterostructure.

Here, the spin-polarized carriers can be either electrons or holes. Generally, the electrons are used as the carriers because the spin lifetime of electrons is much longer than that of holes.

The two-dimensional structure 10 is configured to emit light in response to carrier injection. The light emitted by the two-dimensional structure 10 has a circular polarization state determined by the magnetization state of the spin injector 20.

Two-dimensional (2D) materials, such as graphene, transition metal dichalcogenides (TMDCs like MoS₂, WSe₂ WS₂, or MoSe₂, or an alloy of any two or more of WS₂, WSe₂, MoS₂ and MoSe₂), and hexagonal boron nitride (hBN), have revolutionized materials science due to their atomic-scale thickness and exceptional electronic, optical, and mechanical properties. Unlike conventional 3D materials, 2D materials consist of single or few layers of atoms held together by strong in-plane covalent bonds, while weak Van der Waals (VdW) forces act between layers. This unique structure enables the exfoliation or synthesis of ultra-thin sheets and their reassembly into customized heterostructures.

A 2D van der Waals heterostructure is created by vertically or horizontally stacking different 2D materials without requiring lattice matching. The weak interlayer VdW interactions allow integration of disparate materials, such as combining conductive graphene with semiconducting TMDCs or insulating hBN, to design hybrid systems with tailored functionalities. This “Lego-like” assembly breaks the limitations of traditional semiconductors, enabling unprecedented control over electronic band structures, interfacial charge transfer, and light-matter interactions. For instance, graphene-TMDC heterostructures exhibit enhanced photoresponsivity, while moiré superlattices formed by twisted layers (e.g., “magic-angle” graphene) host correlated electronic states like superconductivity.

These heterostructures hold transformative potential in electronics, optoelectronics, and quantum technologies. Applications include ultra-thin transistors, flexible sensors, high-efficiency solar cells, and quantum emitters. Additionally, their mechanical flexibility and transparency make them ideal for wearable devices.

Hereinafter, more details of the spin light emitting device based on two-dimensional material will be described with reference to FIGS. 2-5.

FIG. 2 is a cross-sectional view of the light emitting device (2D spin-LED) according to an embodiment of the disclosure. The embodiment shown in FIG. 2 is a 2D spin-LED.

FIG. 3 is a top view of the 2D-spin LED according to the embodiment of the disclosure.

In the embodiment, as shown in FIGS. 2 and 3, the two-dimensional structure 10 includes three thin layers: a bottom barrier layer 11, a monolayer 12 of transition metal dichalcogenides and a top barrier layer 13.

The bottom barrier layer 11 is configured to act as a bottom tunneling barrier.

The monolayer 12 of transition metal dichalcogenides is arranged on the bottom barrier layer 11 and possesses direct band gap for light emitting. Specifically, the monolayer 12 of transition metal dichalcogenides generates light in response to the carriers injected from the spin injector 20.

The top barrier layer 13 is arranged on the monolayer 12 of transition metal dichalcogenides and is configured to act as a top tunneling barrier. The spin injector 20 is formed on or above the top barrier layer 13.

In some embodiments, the transition metal dichalcogenides is composed of any one of WS₂, WSe₂, MoS₂ and MoSe₂, or an alloy of any two or more of WS₂, WSe₂, MoS₂ and MoSe₂.

In some embodiments, the bottom barrier layer 11 is composed of hexagonal boron nitride or Al₂O₃.

In some embodiments, the top barrier layer 13 is composed of hexagonal boron nitride or Al₂O₃.

In the embodiment that the top barrier layer 13 is composed of hexagonal boron nitride or Al₂O₃, the spin injector 20 can be deposited on the top barrier layer 13 by using in-situ mask.

In the embodiment that the top barrier layer 13 is composed of Al₂O₃, the spin injector 20 can be formed on the top barrier layer 13 by using UV lithography.

The circular polarization state of the light emitted by the two-dimensional structure 10 is determined by the spin polarization state of the injected carriers. And the spin polarization state of the carriers injected from the spin injector 20 into the two-dimensional structure 10 is determined by the magnetization direction of the spin injector 20.

Therefore, by changing the magnetization direction of the spin injector 20 through the magnetization controller 30, the circular polarization state of the emitted light can be switched accordingly.

In some embodiments, the spin injector 20 is in a form of a bar-shaped channel.

Accordingly, the magnetization controller 30 may include a first electrode 31 and a second electrode 32.

In some embodiments, the magnetization controller 30 may also include a current pulse generator 40. In some embodiments, the current pulse generator 40 is a supplier external to the magnetization controller 30 and applying current pulses to the first electrode 31 and the second electrode 32.

The current pulse generator 40 provides the current pulses with a direction corresponding to the circular polarization direction of the light beam desired to be emitted. And the current pulse generator 40 is capable of alternatively reversing the direction of the current pulses to change the circular polarization direction of the emitted light beam.

The first electrode 31 and the second electrode 32 are respectively connected to two output terminals of a current pulse supplier to receive current pulses to apply current pulse into the bar-shaped channel (spin injector 20) to electrically control the magnetization direction of the spin injector 20.

The first electrode 31 and the second electrode 32 are respectively connected to two opposite ends of the bar-shaped channel of the spin injector 20 to apply a current pulse into the bar-shaped channel, so as to change the magnetization direction of the spin injector 20. With the two electrodes, alternating reverse pulsed current may be applied into the bar-shaped channel to alternatively reverse the magnetization direction of the spin injector 20.

As shown in FIGS. 2 and 3, the first electrode 31 and the second electrode 32 are respectively connected to the two opposite ends in the lengthwise direction of the bar-shaped channel (spin injector 20), so as to introduce the current pulse into the bar-shaped channel to flow through the lengthwise direction.

As shown in FIG. 3, a plurality of bar-shaped spin injectors 20 can be formed above a two-dimensional structure 10. The plurality of spin injectors 20 can be respectively controlled by their respective electrodes (the first electrode 31, the second electrode 32 and the third electrode 33).

The spin injector 20 is configured such that its magnetization direction is capable of being switched by applying a current pulse with a direction opposite to that of the previous current pulse applied into the spin injector 20.

The direction of the current pulse applied into the bar-shaped channel is capable of being reversely switched, for example, by reversing the direction of the current pulses provided by the current pulse generator 40.

In some embodiments, the spin injector 20 is ferromagnetic with out-of-plane magnetization.

In some embodiments, the spin injector 20 is configured to generate spin-orbit torque (SOT).

The spin injector 20 may have a Hall-bar structure, and the magnetization direction of the spin injector 20 can be switched by spin Hall effect (SHE).

In an embodiment, the spin injector 20 is composed of a ferromagnet (FM) layer and a layer for generating spin-orbit torque. The layer for generating spin-orbit torque is composed of a heavy metal (HM) or topological insulators or orbital torque materials.

In a further embodiment, the spin injector 20 is composed of a layer of CoFeB or Fe₃GaTe₂ (ferromagnet (FM)), a layer of Ta or W or Pt (heavy metal (HM)), and a layer of Cr or Ti. The layer of CoFeB may be an ultrathin layer of about 1.1 nm.

The material of CoFeB in the spin injector 20 can also be replaced by ferromagnetic 2D materials (e.g. Fe₃GaTe₂, Crl₃), which has been recently demonstrated a good SOT switching property. The heavy metal can also choose Pt with a relatively large spin Hall angle.

As shown in FIG. 2, a bottom electrode 35 is arranged below the two-dimensional structure 10. The two-dimensional structure 10 is sandwiched between bottom electrode 35 and the spin injector 20.

A third electrode 33 is connected to the spin injector 20. A fourth electrode 34 is connected to the bottom electrode 35,

The third electrode 33 and the fourth electrode 34 are configured to apply a voltage between the spin injector 20 and the bottom electrode 35 to inject spin-polarized carriers from the spin injector 20 into the two-dimensional structure 10.

The third electrode 33 and the fourth electrode 34 are respectively connected to two output terminals of a voltage source supplier 50 to receive voltage signals.

In some embodiments, the first electrode 31 and/or the second electrode 32 may also serve as the third electrode 33 to receive the bias voltage with respect to the fourth electrode 34. In other words, the first electrode 31 and/or the second electrode 32 can be further connected to one output terminal of the voltage source supplier 50 to receive the voltage signals, in addition to receiving the current pulses.

As shown in FIG. 2, the third electrode 33 and the first electrode 31 share the same electrode. The first electrode 31 further serves as the third electrode 33, and is connected to the voltage source supplier 50 to apply voltage signal to the spin injector 20.

In some embodiments, the first electrode 31, the second electrode 32, the third electrode 33 and the fourth electrode 34 are formed from Ti, or Au or combination of Ti and Au such as double-layer film (Ti/Au) or Ti—Au alloy.

By applying the current pulse into the spin injector 20 via the first electrode 31 and the second electrode 32, the magnetization state (magnetization direction) of the spin injector 20 will be changed accordingly.

And then, by applying a bias voltage (first voltage) between the spin injector 20 and the bottom electrode 35 via the third electrode 33 and the fourth electrode 34, spin-polarized carriers will be injected from the spin injector 20 into the two-dimensional structure 10.

In response to the spin-polarized carriers, the two-dimensional structure 10 emits light with circular polarization (σ+ or σ−) corresponding to the spin polarization of the carriers.

And thus, the two-dimensional structure 10 will emit a light beam 100 with right-handed circularly polarized portions (o+) or left-handed circularly polarized portions (σ−).

Pulsed current will be sent into the spin injector 20 via the first electrode 31 and the second electrode 32 to switch the magnetization state of spin injector 20. The spin injector 20 will be negatively biased by the third electrode 33 and the fourth electrode 34 for a continuous emission. σ+ and σ− will be modulated according to the pulsed-current direction in the channel of spin injector 20.

To sum up, the magnetization state of the spin injector 20 can be electrically controlled by applying current pulse into the spin injector 20 via the first electrode 31 and the second electrode 32, the spin direction of the spin-polarized carriers injected into the two-dimensional structure 10 is thus determined, and accordingly, the circular polarization direction of the light emitted by the light emitting device is determined. In other words, the circular polarization direction of the light emitted by the light emitting device can be alternated by changing the direction of the current pulse applied between the first electrode 31 and the second electrode 32 and flowing through the spin injector 20.

To electrically control the circular polarization direction of the emitted light beam, a current pulse will be applied to the spin injector 20 (through the first electrode 31 and the second electrode 32) to switch the magnetization state of the spin injector 20. Then, the spin injector 20 is negatively biased (through the third electrode 33 and the fourth electrode 34) to enable a light emission.

As the magnetization state changes in response to the applied current pulse, the spin polarization state of the carriers injected from the spin injector 20 into the two-dimensional structure 10 also changes accordingly.

According to the emission principle of valley excitons in 2D TMDC material, the circular polarization direction (right circular polarization σ+ or left circular polarization σ−) of the light generated from the two-dimensional structure 10 will be determined by the spin polarization direction of carriers injected from the spin injector 20. Therefore, by switching the magnetization state of the spin injector 20, the spin polarization direction of the injected carriers can be changed, and the circular polarization direction of the generated light beam will be controlled accordingly. The emission principle of valley excitons in 2D TMDC material will be described later with reference to FIG. 7.

In embodiments, the magnetization state of the spin injector 20 refers to the magnetization direction of the spin injector 20.

Magnetization direction of the spin injector 20 is flipped by applying the current pulse into the bar-shaped channel of the spin injector 20.

The magnetization state of the spin injector 20 are non-volatile and are capable of being retained after the current pulse are applied.

The parameters of the current pulse, such as amplitude, duration, numbers of sub-pulse and so on, can be configured to make sure that the magnetization direction of the whole spin injector 20 is flipped into one magnetization direction, for example, up-direction (⬆).

While a bias voltage is applied between the third electrode 33 and the fourth electrode 34, spin-polarized carriers are injected from the spin injector 20 to the two-dimensional structure 10.

When the spin injector 20 has an up-direction (⬆) magnetization, the majority of carriers injected from the spin injector 20 into the two-dimensional structure 10 are polarized to spin of +½. Accordingly, the light generated from the two-dimensional structure 10 has a positive circular polarization, in which the proportion of the right circularly polarized light (σ+) component is greater than that of the left circularly polarized light component (σ−).

And, when the spin injector 20 has a down-direction (⬇) magnetization, the majority of carriers injected from the spin injector 20 into the two-dimensional structure 10 are polarized to spin of −½. Accordingly, the light generated from the two-dimensional structure 10 has a negative circular polarization, in which the proportion of the right circularly polarized light (σ+) component is smaller than that of the left circularly polarized light component (σ−).

In addition, after a light beam is generated from the two-dimensional structure 10 with the spin injector 20 in one magnetization direction, the magnetization direction of the spin injector 20 can be flipped to the opposite direction, for example, from up-direction (1) magnetization to down-direction (⬇) magnetization, by applying a further current pulse with a direction opposite to the previous one.

As shown in FIG. 2, the bottom electrode 35 may be prepatterned to be surrounded by an insulating layer 60, and the bottom electrode 35 and the insulating layer 60 is formed on a substrate 70.

In an embodiment, the bottom shown in FIG. 2 is composed of ITO, the insulating layer 60 is composed of SiO₂, and the substrate 70 is composed of glass.

A specific example of the 2D spin-LED shown in FIGS. 2 and 3 will be described in detail hereinafter.

The 2D spin-LED includes two parts. One part is 2D VdW heterostructure (the two-dimensional structure 10) for emitting light. One example of the two-dimensional structure 10 could be a three-layer structure: a layer of hexagonal boron nitride (BN), a layer of WS₂ or WSe₂ or MoS₂ or MoSe₂, or their any alloy, and a layer of BN, which can be fabricated by different techniques, such as MBE or MOCVD growth, exfoliation with dry transfer, or 2D wet transfer techniques.

The top BN layer 13 and bottom BN layer 11 have a thickness of about 1-2 nm, serving as tunneling barriers.

The middle layer of WS₂ or WSe₂ or MOS₂ or MoSe₂ or their any alloy is monolayer (ML) so that it possesses direct band gap for emitting light.

The other part is consisted of the metallic spin injector 20 on the top of 2D heterostructure 10. The spin injector 20 is of a three-layer structure of ultrathin CoFeB (1.1 nm)/Ta or W/Cr, which magnetization is in out-of-plane direction.

The spin injector 20 has a Hall-bar structure either directly using in-situ mask for deposition, or structured with lithography. The magnetization of spin injector 20 can be switched by spin Hall effect (SHE).

As shown in FIG. 3, large size monolayer (ML) 2D materials (BN, WS₂) will be alternatively transferred on an insulating layer 60 with prepatterned ITO bottom electrode 35, to form the sandwiched BN/WS₂/BN 2D heterostructure 10.

Then the bar shaped top ferromagnetic electrode (CoFeB/Ta/Cr) (spin injector 20) will be deposited on the 2D structure 10 by using in-situ mask. A voltage between the third electrode 33 and the further electrode 34 will be applied to emit the light.

The spin polarized electrons injected from CoFeB/Ta/Cr spin injector 20 will recombine with the opposited polarized holes in one of the K and −K valleys due to the spin-valley locking and emit the circularly polarized light. The circular polarization of light depends on the injected spin polarization.

To demonstrate the capability of electrical control of Pc, pulsed current will be sent into the bar-shaped channel of the spin injector 20 through the first electrode 31 and the second electrode 32, to switch the magnetization direction of the spin injector 20. The circular polarization Pc (σ+ and σ−) of the emitted light will be modulated according to the pulsed current direction applied in the bar-shaped channel of the spin injector 20.

Since the size resolution of in-situ mask is limited to 50 μm, another method to form the Hall bar structure of the spin injector 20 is using UV lithography. In that case, the top BN barrier 13 will be replaced by Al₂O₃ layer (2-3 nm), which can be grown by atomic layer deposition (ALD). The CoFeB/Ta/Cr layer for forming the spin injector 20 will be deposited in the whole surface and the bar-shaped structure of the spin injector 20 will be formed by UV lithography. The reason to replace BN by Al₂O₃ is that the Al₂O₃ layer can better protect the 2D surface by preventing from the damage during ion milling process.

In a variant embodiment, the monolayer 12 of the two-dimensional structure 10 can be substituted by a plurality of monolayer groups.

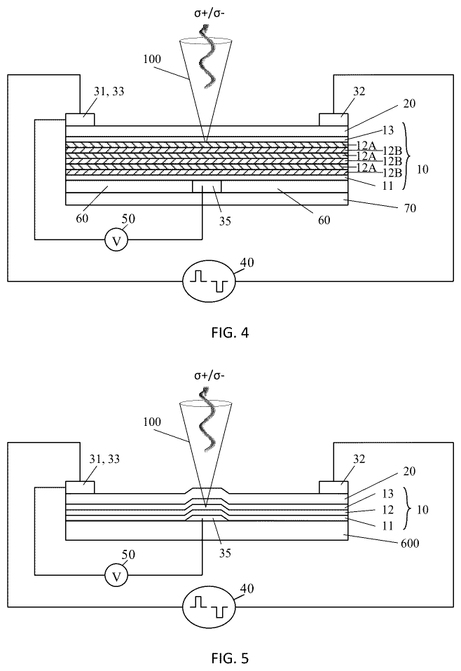

FIG. 4 is a cross-sectional view of the light emitting device (2D spin-LED) according to a variant embodiment of the disclosure, in which a plurality of monolayer groups are introduced.

As shown in FIG. 4, a plurality of monolayer groups are arranged on the bottom barrier layer 11 and are configured to possess direct band gap for emitting light.

Each monolayer group includes more than one monolayer. Adjacent monolayers are composed of different transition metal dichalcogenides, and the monolayers in the plurality of monolayer groups are arranged in the same sequence. The transition metal dichalcogenides of the respective monolayer might be composed of any one of WS₂, WSe₂, MoS₂ and MoSe₂, or an alloy of any two or more of WS₂, WSe₂, MoS₂ and MoSe₂.

The band gap of adjacent monolayer is different. The plurality of monolayer groups may form a multiple quantum well (MQW) structure. Accordingly, the light emission efficiency might be improved.

In the example shown in FIG. 4, three monolayer groups are sandwiched by the bottom barrier layer 11 and the top barrier layer 13. Each monolayer groups includes two monolayers 12A and 12B. The two monolayers 12A and 12B are composed of different transition metal dichalcogenides.

It shall be understood that there might be two, three or more than three monolayer groups stacked in the two-dimensional structure 10, and each monolayer group may include two more than two monolayers. The monolayer groups form a periodic stacking of multiple monolayers of different transition metal dichalcogenides.

For example, the plurality of monolayers may be a periodic stacking of monolayers of four kind of different transition metal dichalcogenides, e.g., A, B, C and D. The periodic stacking of monolayers might be ABCDABCDABCD, where ABCD is a monolayer group.

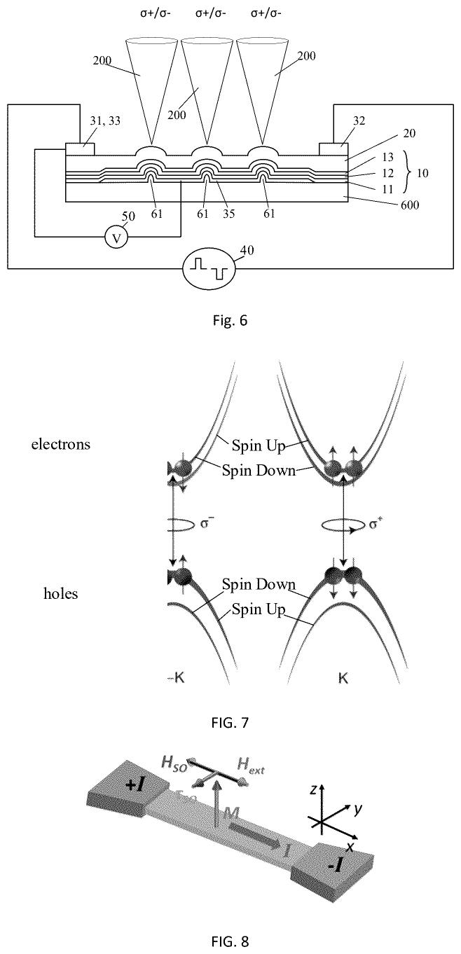

It shall be understood that the monolayer 12 to be described below with reference to FIG. 5 and FIG. 6 may also be substituted by a plurality of monolayer groups as described above with reference to FIG. 4.

FIG. 5 is a cross-sectional view of the light emitting device (2D spin-LED) according to another variant embodiment of the disclosure.

FIG. 5 shows another possibility is to replace the ITO material of bottom electrode 35 by graphene (Gr). Graphene (Gr) electrode has a high mobility and better matching of Fermi level with conduction band of WS₂.

In this case, after transfer of a graphene (Gr) layer to the SiO₂ substrate 60, UV lithography and oxygen plasma etching will be performed to form the bar shape bottom electrode 35. There is no need to fill SiO₂ to flatten the surface as the case of ITO shown in FIG. 2.

FIG. 6 is a cross-sectional view of the light emitting device (2D spin-SPS) according to yet another variant embodiment of the disclosure. The embodiment shown in FIG. 6 is an electrically pumped two-dimensional spin-based Single Photon Source (2D spin-SPS). The 2D spin-SPS shown in FIG. 6 is developed based on the structure of 2D spin-LED shown in FIGS. 2-4.

As shown in FIG. 6, one or a plurality of nanopillars 61 are formed on an upper surface of the substrate 60.

In some embodiments, the plurality of nanopillars are arranged in an array.

The two-dimensional structure 10 is formed above the upper surface with protrusions generated at the nanopillar 61 positions. And, the bottom electrode 35 is sandwiched between the substrate and the two-dimensional structure.

The other features of the spin-SPS may be the same as or similar to those of the spin-LED, and can be formed by the same or similar process as those of the spin-LED.

For example, similar to the 2D spin-LED shown in FIGS. 2-4, the two-dimensional structure 10 may include three thin layers: a bottom barrier layer 11, a monolayer 12 of transition metal dichalcogenides and a top barrier layer 13. The three layers may be composed of the same materials as those in the 2D spin-LED as described above with reference to FIGS. 2-4.

Further, similar to the 2D spin-LED, the 2D spin-SPS has a spin injector 20 formed above the two-dimensional structure 10. The spin injector 20 may be a bar-shaped channel. And the spin injector 20 of the 2D spin-SPS may be composed of the same materials as those of the spin injector 20 of the 2D spin-LED. The spin injector 20 is ferromagnetic with out-of-plane magnetization. In some embodiments, the spin injector 20 is configured to generate spin-orbit torque (SOT). In some embodiments, the spin injector is composed of a ferromagnet layer and a layer for generating spin-orbit torque, the layer for generating spin-orbit torque is composed of heavy metal or topological insulators or orbital torque materials. In some embodiments, the spin injector 20 might be free of metal. The spin injector 20 may have a Hall-bar structure, and the magnetization direction of the spin injector 20 can be switched by spin Hall effect (SHE).

Further, similar to the 2D spin-LED, the 2D spin-SPS may have the first electrode 31 and the second electrode 32 being connected to the two opposite ends of the bar-shaped channel of the spin injector 20 to apply a current pulse into the bar-shaped channel, so as to change the magnetization direction of the spin injector 20.

As described above, the spin polarization state of the carriers injected from the spin injector 20 into the two-dimensional structure 10 is determined by the magnetization direction of the spin injector. And, the circular polarization state of the single photons emitted by the two-dimensional structure 10 is determined by the spin polarization state of the injected carriers.

And, the 2D spin-SPS may have the bottom electrode 35. The two-dimensional structure 20 is sandwiched between the bottom electrode 35 and the spin injector 20. The third electrode 33 is connected to the spin injector 20 and the fourth electrode 34 is connected to the bottom electrode. The third electrode 33 and the fourth electrode 34 are configured to apply a voltage between the spin injector 20 and the bottom electrode 35 to inject spin-polarized carriers from the spin injector 20 into the two-dimensional structure 10. The third electrode 33 may share the same electrode with the first electrode 31 and/or the second electrode 32.

The light emitting device shown in FIG. 6 is thus a two-dimensional spin-based single photon source, single photons 200 will be emitted at the protrusions in response to carrier injection.

A specific example of the 2D spin-SPS shown in FIG. 6 will be described in detail hereinafter.

The 2D geometry of a single photon sources confined to an atomically thin material can greatly enhance the photon extraction efficiency, allowing for simplified integration with photonic circuits, and could facilitate strong and controllable external perturbations due to the close proximity of the embedded SPS.

With the spin injector 20, the 2D spin-SPS can emit circularly polarized single photons 200, this is extremely important to obtain indistinguishable single photons 200 for quantum communication and computation applications.

In addition, spin dependent quantum emission in 2D materials 10 could also be used to initialize or read out spin valley information from another neighboring 2D materials.

To create quantum photon emitters, we can use nanopillar (61) array with precise position since the nanopillars (61) can create localized deformations which lead to the quantum confinement of excitons.

By placing the 2D materials (two-dimensional structure 10) on spatially ordered nanopillars 61, the position of single photon emission can be controlled.

The substrate 600 may be composed of silica (SiO₂). Silica (SiO₂) is a convenient substrate material for making nanopillars 61, which can be fabricated by electron beam lithography. The nanopillars 61 may be formed by the same material as the substrate 600, i.e. silica (SiO₂).

By electrically switching of the magnetization direction of the spin injector 20, the circular polarization state of emitted single photons 200 can be controlled.

The emission characteristics of single photons 200 are dependent on the height of nanopillars 61. For the taller nanopillars, the linewidth of the emission peaks will be narrower. However, if it is too tall, the 2D material 10 will be pierced by the nanopillar 61. Therefore, it is important to optimize the quality of single photons 200 by tuning the height of nanopillars 61.

In addition, the nanopillar 61 could also influence on the magnetic domain propagation in the bar-shaped channel of the spin injector 20. Too high nanopillar could pin the magnetic domain of the spin injector 20 and prevent it from efficient SOT switching.

The spin light emitting devices based on two-dimensional materials of the disclosure is described in detail above.

In embodiments of the disclosure, by using SOT spin injector on 2D LED and SPS, it is possible to control the spin injection and consequently it is possible to modulate the circular polarization of emitted light. There are several advantages for such CoFeB SOT spin injector.

CoFeB has a high Curie temperature (980K) and with a high spin polarization (60%), therefore it is possible to achieve high spin injection at room temperature.

The spin injector possesses perpendicular magnetic anisotropy, which allows to emit circularly polarized light from spin-LED and spin-SPS without external applied magnetic field.

The total thickness of the spin injector layer is very thin (about 5 nm), ensuring a low optical absorption loss (<10%) and a small Magneto-Circular Dichroism (MCD) effect (<1%).

Since the CoFeB injector magnetization switching time can be less than 5 ps, it allows ultrafast modulation of the circular polarization in a range of about 100 GHz.

Therefore, the spin light emitting device based on two-dimensional materials can fulfill the three conditions: room temperature, no need of field and electrical control, which paves the way for practical applications.

Hereinafter, the emission principle of valley excitons in two-dimensional TMDC materials and the magnetization switching in the spin injector will be described with reference to FIGS. 6 and 7.

FIG. 7 schematically shows the emission principle of valley excitons in two-dimensional TMDC materials.

The transition metal dichalcogenides (TMDCs) based 2D monolayers (e.g. WSe₂, WS₂, MoSe₂, MoS₂ . . . , or their any alloy) possess direct energy gaps located at the unequal-K and K valleys in the reciprocal space, as shown in FIG. 7.

The spin degeneracy at the valence band edges is lifted by spin-orbit interactions. Electrical excitation and confinement of the carriers in one set of the two non-equivalent valleys are achieved through the manipulation of the injected carrier spin polarizations, due to the spin-valley locking in monolayer TMDCs.

The electron-hole recombination in the −K and K valleys performs opposite EL helicity. In FIG. 7, the spin-down bands and spin-up bands respectively for electrons and holes of TMDC (e.g. WSe₂) are indicated. The spin-orbit splitting in the valence band (˜450 meV, curves drawn in the lower half of FIG. 7, for spin-polarized holes) and conduction band (˜25 meV, curves drawn in the upper half of FIG. 7, for spin-polarized electrons) are sketched disproportionally for clarity.

Optical selection rules give rise to opposite circularly polarized light emissions at different excited valleys.

If 100% spin down electrons is injected in-K valley, 100% left-handed circularly polarized light will be emitted.

If 100% spin up electrons is injected in K valley, 100% right-handed circularly polarized light will be emitted.

The spin up electrons in-K valley cannot combine with spin down hole because of spin-orbit generated splitting of valence band.

The spin down electrons in K valley cannot combine with spin up hole because of spin-orbit generated splitting of valence band.

Therefore, the circular polarization direction of the single photons 200 emitted from two-dimensional structure completely depends on the spin direction of the injected electron. And thus, the circular polarization direction of the light emitted from the two-dimensional structure 10 corresponds to the spin polarization direction of the carriers injected into the two-dimensional structure 10.

It should be emphasized here that the above optical selection rule requires the spin direction to be parallel to the photon emission direction. To obtain circularly polarized single photons 200 without magnetic field, the magnetization direction of the ferromagnetic injection layer (spin injector 20) shall be perpendicular to the sample surface for surface emission geometry.

Based on the above principle, the two-dimensional structure 10 is capable of emitting light beam with controllable circular polarization direction by injecting carriers with controlled spin polarization direction into the two-dimensional structure 10.

FIG. 8 illustrates the magnetization switching in the spin injector with a Hall-bar structure by SHE.

As shown in FIG. 8, a current/is injected in the designed ferromagnet (FM)/heavy metal (HM) spin-injector channel to generate current-induced spin-orbit torque (SOT) τₛₒ and associated spin-orbit field Hₛₒ. With a small in-plane external constant magnetic field Hₑₓₜ, the perpendicular magnetization of injector can be deterministically switched when injecting alternative direction of current in the channel. The magnetization switch can be realized with very short pulse current (6 ps), which allows for high-speed operation. The latest developments in the field of spintronics show the possibility to avoid the application of Hₑₓₜ by using different strategies, such as using spin textured ferromagnetic layer, an in-plane exchange bias or growth on substrates with specific crystalline orientation.

Those skilled in the art may understand that appropriate modifications can be made to various above-described light emitting device structures of the present disclosure as needed, all of which are within the scope of protection of the present disclosure.

Various embodiments of the present disclosure have been described above, and the foregoing descriptions are exemplary, not exhaustive, and not limiting of the disclosed embodiments. Numerous modifications and variations will be apparent to those of ordinary skill in the art without departing from the scope and spirit of the described embodiments. The terminology used herein was chosen to best explain the principles of the various embodiments, the practical application or improvement over the technology in the marketplace, or to enable others of ordinary skill in the art to understand the various embodiments disclosed herein.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A light emitting device comprising:
a two-dimensional structure configured to emit light in response to carrier injection, wherein the two-dimensional structure is a two-dimensional Van der Waals heterostructure;
a spin injector configured to inject spin-polarized carriers into the two-dimensional Van der Waals heterostructure, wherein the light emitted by the two-dimensional structure has a circular polarization state determined by the magnetization state of the spin injector; and
a magnetization controller configured to change the magnetization state of the spin injector.

2. The light emitting device according to claim 1, wherein the two-dimensional structure comprises:
a bottom barrier layer configured to act as a bottom tunneling barrier;
a monolayer of transition metal dichalcogenides arranged on the bottom barrier layer and configured to possess direct band gap for emitting light; and
a top barrier layer arranged on the monolayer of transition metal dichalcogenides and configured to act as a top tunneling barrier.

3. The light emitting device according to claim 1, wherein the two-dimensional structure comprises:
a bottom barrier layer configured to act as a bottom tunneling barrier;
a plurality of monolayer groups arranged on the bottom barrier layer and configured to possess direct band gap for emitting light, each monolayer group comprises more than one monolayers, adjacent monolayers are composed of different transition metal dichalcogenides, and the monolayers in the plurality of monolayer groups are arranged in the same sequence; and
a top barrier layer arranged on the plurality of monolayer groups and configured to act as a top tunneling barrier.

4. The light emitting device according to claim 2, wherein
the transition metal dichalcogenides is composed of any one of WS₂, WSe₂, MoS₂ and MoSe₂, or an alloy of any two or more of WS₂, WSe₂, MoS₂ and MoSe₂; and/or
the bottom barrier layer is composed of hexagonal boron nitride or Al₂O₃; and/or
the top barrier layer is composed of hexagonal boron nitride or Al₂O₃.

5. The light emitting device according to claim 3, wherein
the top barrier layer is composed of hexagonal boron nitride or Al₂O₃, and the spin injector is deposited on the top barrier layer by using in-situ mask; or
the top barrier layer is composed of Al₂O₃, and the spin injector is formed on the top barrier layer by using UV lithography.

6. The light emitting device according to claim 1, wherein the spin injector is in a form of a bar-shaped channel, the magnetization controller comprises:
a first electrode and a second electrode respectively connected to two opposite ends of the bar-shaped channel to apply a current pulse into the bar-shaped channel, so as to change the magnetization direction of the spin injector,
wherein the spin polarization state of the carriers injected from the spin injector into the two-dimensional structure is determined by the magnetization direction of the spin injector; and
the circular polarization state of the light emitted by the two-dimensional structure is determined by the spin polarization state of the injected carriers.

7. The light emitting device according to claim 6, wherein
the direction of the current pulse applied into the bar-shaped channel is capable of being reversely switched, and
the spin injector is configured such that its magnetization direction is capable of being switched by applying a current pulse with a direction opposite to that of the previous current pulse applied into the spin injector.

8. The light emitting device according to claim 1, wherein
the spin injector is ferromagnetic with out-of-plane magnetization; and/or
the spin injector is configured to generate spin-orbit torque; and/or
the spin injector is composed of a ferromagnet layer and a layer for generating spin-orbit torque, the layer for generating spin-orbit torque is composed of heavy metal or topological insulators or orbital torque materials.

9. The light emitting device according to claim 8, wherein
the spin injector is composed of a layer of CoFeB or Fe₃GaTe₂, a layer of Ta or W or Pt, and a layer of Cr or Ti.

10. The light emitting device according to claim 1 further comprising:
a bottom electrode, the two-dimensional structure is sandwiched between the bottom electrode and the spin injector;
a third electrode connected to the spin injector; and
a fourth electrode connected to the bottom electrode,
wherein the third electrode and the fourth electrode are configured to apply a voltage between the spin injector and the bottom electrode to inject spin-polarized carriers from the spin injector into the two-dimensional structure.

11. The light emitting device according to claim 10, wherein
the bottom electrode is formed from ITO or graphene.

12. The light emitting device according to claim 1, further comprising:
a substrate,
one or a plurality of nanopillars are formed on an upper surface of the substrate,
the two-dimensional structure is formed above the upper surface with protrusions generated at the nanopillar positions, and the bottom electrode is sandwiched between the substrate and the two-dimensional structure.

13. The light emitting device according to claim 12, wherein
the plurality of nanopillars is arranged in an array.

14. The light emitting device according to claim 12, wherein
the light emitting device is a two-dimensional spin-based single photon source, and circularly polarized single photon emission occurs at the protrusions in response to spin-polarized carrier injection.

15. The light emitting device according to claim 12, wherein the two-dimensional structure comprises:
a bottom barrier layer configured to act as a bottom tunneling barrier;
a monolayer of transition metal dichalcogenides arranged on the bottom barrier layer and configured to possess direct band gap for emitting light; and
a top barrier layer arranged on the monolayer of transition metal dichalcogenides and configured to act as a top tunneling barrier.

16. The light emitting device according to claim 15, wherein
the transition metal dichalcogenides is composed of any one of WS₂, WSe₂, MoS₂ and MoSe₂, or an alloy of any two or more of WS₂, WSe₂, MoS₂ and MoSe₂; and/or
the bottom barrier layer is composed of hexagonal boron nitride or Al₂O₃; and/or
the top barrier layer is composed of hexagonal boron nitride or Al₂O₃.

17. The light emitting device according to claim 12, wherein the spin injector is in a form of a bar-shaped channel, the magnetization controller comprises:
a first electrode and a second electrode respectively connected to two opposite ends of the bar-shaped channel to apply a current pulse into the bar-shaped channel, so as to change the magnetization direction of the spin injector,
wherein the spin polarization state of the carriers injected from the spin injector into the two-dimensional structure is determined by the magnetization direction of the spin injector; and
the circular polarization state of the single photons emitted by the two-dimensional structure is determined by the spin polarization state of the injected carriers.

18. The light emitting device according to claim 17, wherein
the direction of the current pulse applied into the bar-shaped channel is capable of being reversely switched, and
the spin injector is configured such that its magnetization direction is capable of being switched by applying a current pulse with a direction opposite to that of the previous current pulse applied into the spin injector.

19. The light emitting device according to claim 12, wherein
the spin injector is ferromagnetic with out-of-plane magnetization; and/or
the spin injector is configured to generate spin-orbit torque; and/or
the spin injector is composed of a ferromagnet layer and a layer for generating spin-orbit torque, the layer for generating spin-orbit torque is composed of heavy metal or topological insulators or orbital torque materials.

20. The light emitting device according to claim 12 further comprising:
a bottom electrode, the two-dimensional structure is sandwiched between the bottom electrode and the spin injector;
a third electrode connected to the spin injector; and
a fourth electrode connected to the bottom electrode,
wherein the third electrode and the fourth electrode are configured to apply a voltage between the spin injector and the bottom electrode to inject spin-polarized carriers from the spin injector into the two-dimensional structure.
